(19) United States
(12) Patent Application Publication
(10) Pub. No.: US 2012/0203028 A1
(43) Pub. Date: Aug. 9, 2012

(54) PROCESS FOR THE PREPARATION OF PURE ACRYLATE ESTERS

(75) Inventors: Dov Beruben, Beer Sheva (IL); Ofra Yosef, Beer Sheva (IL); Michael Peled, Beer Sheva (IL)

(73) Assignee: BROMINE COMPOUNDS LTD., Beer Sheva (IL)

(21) Appl. No.: 13/499,708

(22) PCT Filed: Sep. 7, 2010
(86) PCT No.: PCT/IL10/00736

§ 371 Date: Apr. 2, 2012

(30) Foreign Application Priority Data
Oct. 15, 2009 (IL) 201584

(51) Int. Cl. C07C 67/287

(52) U.S. Cl. 560/223

(57) ABSTRACT

The present invention provides a process for manufacturing acrylate esters of trinol, dinol, and didinol, which avoids the undesired thermopolymerization of the esters and provides highly pure monomers for the preparation of plastic lenses.

### FIELD OF THE INVENTION

The present invention relates to the preparation of acrylate esters, and particularly to a process of manufacturing acrylate esters of 3-bromo-2,2-bis (bromomethyl)propane -1-ol (trinol), 2,2-bis(bromomethyl)propane-1,3-diol (dinol), and 3,3′-oxybis(2,2-bis(bromomethyl)propan-1-ol)(didinol), comprising inhibiting the undesired thermopolymerization of said esters.

### BACKGROUND OF THE INVENTION

Plastic lenses are now widely used beside inorganic glasses, because of their low weight, workability, stability, dyeability, and low cost. Of the physical properties required for lenses, a high refractive index (RI) and a low dispersion (high Abbe's number) are extremely important. JP5828117 and JP5754901 employ halogen-substituted bisphenol A derivatives as monomers in the production of halogen-containing lenses. WO 07/007332 discloses the use of tribromoneopentyl acrylate or methacrylate as monomers for the preparation of plastic lenses. These compounds copolymerize with multifunctional acrylates to form copolymers which may be very useful for the production of lenses in view of their optical and mechanical properties. The acrylate esters may be prepared easily by standard esterification method, wherein the appropriate alcohol is esterified with the desired carboxylic acid in the presence of suitable esterification catalyst and polymerization inhibitors. The use of pure monomers is critical for achieving good results; however, availability of highly pure monomers has been a problem. U.S. Pat. No. 3,480,600 relates to the reaction of 3-bromo-2,2-bis(bromomethyl)propane-1-ol (tribromoneopentyl alcohol or FR-513, commercially available as trinol) with acrylic acid. The monomer was then purified by distillation at reduced pressure (b.p. of 138-141° C. at 1.23-3 mm Hg). Above mentioned WO 07/007332 performed the reaction in toluene in the presence of sulfuric acid as catalyst. The resulting crude monomer was distilled at reduced pressure, at 150° C. However, these high temperatures cause thermopolymerization, resulting in quite a low yield of the monomer, and further undesired discoloration of the obtained monomer. It is therefore an object of the invention to provide a means for preventing the undesirable thermopolymerization during the manufacture of acrylate esters.

It is a further object of the invention to provide a method of manufacturing pure monomers of acrylate ester derivatives.

Other objects and advantages of present invention will appear as description proceeds.

### SUMMARY OF THE INVENTION

The present invention provides a process for manufacturing pure monomers essentially consisting of esters of acrylic acid derivatives and 2,2-bis(bromomethyl)propanol derivatives of formula (I) (BBMP derivatives)

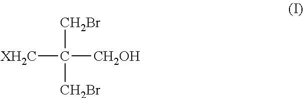

wherein

X is selected from Br, OH, and HOC(CH₂Br)₂O;
comprising i) providing a BBMP derivative, and a derivative of acrylic acid; ii) reacting said BBMP derivative with said acrylic acid derivative in a mixture comprising a first organic solvent (reaction solvent), an acid catalyst, and a polymerization inhibitor, while removing water formed during the reaction; iii) neutralizing said mixture with an aqueous base; iv) separating organic phase from said mixture; v) removing said reaction solvent from said organic phase by evaporation, thereby obtaining a crude oily product; vi) chromatographying said crude product, preferably on silica, employing a second organic solvent (chromatography solvent), which could be the same or different from said reaction solvent; and vii) removing said second solvent;

thereby obtaining said monomers, having a purity of at least 95%. Said BBMP derivatives are selected from the group consisting of 3-bromo-2,2-bis(bromomethyl)propane-1-ol; 2,2-bis(bromomethyl)propane-1,3-diol; and 3,3′-oxybis(2,2-bis(bromomethyl) propan-1-ol). Said acrylic acid derivative is selected from acrylic acid, methacrylic acid, acryloyl halides. Said ester monomer may comprise (meth)acrylate monoester or (meth)acrylate diester. Said ester monomers may be, for example, selected from 3-bromo-2,2-bis(bromomethyl)propyl acrylate; 3-bromo-2,2-bis(bromomethyl)propyl methacrylate; 2,2-bis(bromomethyl)propane-1,3-diyldiacrylate; 2,2-bis(bromomethyl)propane-1,3-diylbis(2-methylacrylate); 3,3′-oxybis(2,2-bis(bromomethyl)propane-1-yl) diacrylate; and 3,3′-oxybis(2,2-bis(bromomethyl)propane-1-yl) bis(2-methylacrylate). Said step (ii) may comprise azeotropic distillation. Said reaction solvent may be selected from the usual solvents for esterification, for example, cyclohexane, or toluene. Said chromatography solvent may be selected from the nonpolar solvents, for example cyclohexane or toluene. In a preferred embodiment of the invention, the product of said step (v) in the described process is subjected to at least one repeated flash chromatography. Said catalyst may be, for example, sulfuric acid. Said polymerization inhibitor may be selected for example from p-methoxyphenol, hydroquinone, and sulfur.

### DETAILED DESCRIPTION OF THE INVENTION

It has been found that using column chromatography in the purification of crude acrylate esters, as described above, after essentially complete evaporation of solvents from the reaction mixture which comprises an acrylate-ester and solvents, together with eventual residual reactants or side products, provided surprisingly high-purity products. The invention relates to the purification of monoester- or diester monomers comprising a 2,2-bis(bromomethyl) propanol (BBMP) derivative esterified with an acrylic acid derivative. Pure monomers according to the invention may comprise acrylic or methacrylic esters of BBMP, wherein said BBMP derivative may be selected from 3-bromo-2,2-bis (bromomethyl)propane-1-ol (trinol); 2,2-bis(bromomethyl)propane-1,3-diol (dinol); and 3,3′-oxybis(2,2-bis(bromomethyl)propan-1-ol)(didinol). A preferred monoester may comprise trinol esterified by acrylic acid to provide 3-bromo-2,2-bis(bromomethyl)propyl acrylate (trinol acrylate, formula Ia) or by methacrylic acid to provide 3-bromo-2,2-bis(bromomethyl)propyl methacrylate (trinol methacrylate, formula Ib)]. Other preferred monomer may comprise dinol esterified by acrylic acid to provide 2,2-bis(bromomethyl)propane-1,3-diyldiacrylate (dinol diacrylate, formula IIa) or by methacrylic acid to provide 2,2-bis(bromomethyl) propane-1,3-diyl bis(2-methylacrylate) (dinol bismethacrylate, formula IIb). Still other preferred monomer may comprise didinol esterified by acrylic acid to provide 3,3′-oxybis(2,2-bis(bromomethyl)propane-1-yl) diacrylate (didinol diacrylate, formula IIIa) or by methacrylic acid to provide 3,3′-oxybis(2,2-bis(bromomethyl)propane-1-yl) bis(2-methylacrylate) (didinol bismethacrylate, formula IIIb).

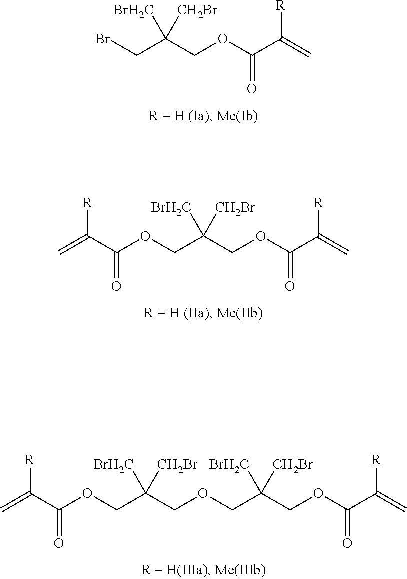

In a typical procedure, the desired alcohol, (e.g. trinol, dinol or didinol), is dissolved in an appropriate solvent, such as toluene or cyclohexane, at an appropriate temperature depending on the used solvent, in the presence of an acid catalyst such as sulfuric acid. Further, a polymerization inhibitor is added to prevent premature and undesired polymerization of the unsaturated compounds possibly utilizing inhibitors known in the field. This inhibitor may be selected from hydroquinone, p-methoxyphenol, or sulfur, etc. (see for example U.S. Pat. No. 3,165,502). The suitable α,β-unsaturated carboxylic acid such as acrylic acid or methacrylic acid is then added dropwise to the reaction mixture. An azeotropic distillation is performed to remove water.

After completion of the reaction, the reaction mixture is treated with aqueous sodium bicarbonate solution in order to bring said mixture to pH=6. Then, reaction mixture is separated from the aqueous solution. The next step includes the removal of the organic solvent of said reaction, to obtain a yellow oil. This oil sample contains a crude monomer which is submitted to a chromatography column, such as a column of silica gel, using a non polar solvent to retain on the column the unreacted starting material for example, cyclohexane or toluene, as eluent. The method of the invention typically provided trinolacrylate or trinolmethacrylate of a purity of about 98% by GC or higher. Due to avoiding elevated temperatures, the thermopolymerization is not observed during the purification of the material, and a high yield is obtained. The final material is only slightly colored. The gentle method according to the invention typically provides a material exhibiting the purity of 98% or more. The monomer manufactured according to the instant method provided polymer materials which were uncolored and transparent, exhibiting the desired refraction index values. The process according to the invention provides superior starting materials for producing polymer lenses.

The invention will be further described and illustrated in the following examples.

### EXAMPLES

The refractive indexes of acrylate derivatives are shown in Tab. 1.

Refractive Index of the acrylate esters derivatives

| Alcohol | Acid | | Esters | form | R.I. |
|---|---|---|---|---|---|
| Trinol | acrylic acid | | trinolacrylate | Liq. | 1.548 |
| | | methacrylic | trinolmethacrylate | Liq. | 1.538 |
| | | acid | | | |
| Dinol | acrylic acid | | Dinol Diacrylate | solid | — |
| Didinol | acrylic acid | | Didinol Diacrylate | Liq. | 1.523 |

### Example 1

### Production of 3-bromo-2,2-bis(bromomethyl)propyl acrylate (Ia)

In a four-neck flask equipped with mechanical stirrer, thermometer, dropping funnel and Dean-Stark apparatus, 490 g (1.5 moles) of trinol were dissolved in 600 ml of cyclohexane followed by the addition of 7.5 ml of sulfuric acid and 0.5 g of sulfur. The reaction mixture was heated to 70° C. Subsequently, 162 g (2.2 moles) of acrylic acid were added dropwise within 1 h and the reaction was stirrer until 27 ml (1.5 moles) of water have accumulated in the Dean-Stark trap. GC analysis showed a conversion of 93-95% (based on peak areas). After cooling to room temperature, 560 ml of an aqueous saturated sodium bicarbonate solution were added until pH=6. After phase separation, the organic phase was concentrated using a rotary evaporator until obtaining ˜580 g of a crude oil material with a purity of about 91% by GC. Purification by chromatography on silica gel (cake of silica gel (370 g, Merck, 0.04-0.063 mm, 230-240 mesh) on a Buechner funnel (2L, Por. 3)) was performed using cyclohexane as eluent. After evaporation of the cyclohexane, 490 g of the expected trinolacrylate was obtained (82% yield) with a purity of 98% by qualitative GC. The column was then washed with ethanol to recover the rest of the material which was submitted, after evaporation of the solvent, to another flash chromatography. 53 g of additional trinol acrylate was obtained with the same purity as previously observed, for an overall yield of 95%.

### Example 2

### Production of 3-bromo-2,2-bis(bromomethyl)propyl methacrylate (Ib)

The process of above Example 1 was repeated except that an equivalent amount of methacrylic acid was used in place of acrylic acid. The crude material was submitted to a flash chromatography, with an improvement of the purity from 86% before purification to 97%.

### Example 3

### Production of 2,2-bis(bromomethyl)propane-1,3-diyl diacrylate (IIa)

The process of above Example 1 was repeated except that the corresponding amount of acrylic acid was reacted with dinol instead of trinol. The crude material was submitted to a flash chromatography, with an improvement of the purity from 86% before purification to 97%.

### TGA: 164° C. (2%); 187° C. (5%); 205° C. (10%).

### Example 4

### 3,3′-oxybis(2,2-bis(bromomethyl)propane-1-yl) diacrylate (IIIa)

The process of above Example 3 was repeated except that the corresponding amount of acrylic acid was reacted with didinol instead of dinol. The crude material was submitted to a flash chromatography, with an improvement of the purity from 87% before purification to 95%

### TGA: 204° C. (2%); 296° C. (5%); 350° C. (10%).

The Refractive Index of the products was measured using a Reichert Abbe Mark II refractometer and the results are summarized in table 1.

While this invention has been described in terms of some specific examples, many modifications and variations are possible. It is therefore understood that within the scope of the appended claims, the invention may be realized otherwise than as specifically described.

1. A process of manufacturing pure monomers essentially consisting of esters of acrylic acid derivatives and 2,2-bis(bromomethyl)propanol derivatives, said 2,2-bis(bromomethyl)propanol derivatives having formula I 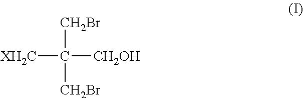
wherein X is selected from Br, OH, and HOC(CH₂Br)₂O; comprising
i) providing a 2,2-bis(bromomethyl)propanol (BBMP) derivative, and a derivative of acrylic acid;
ii) reacting said BBMP derivative with said acrylic acid derivative in a mixture comprising a first organic solvent (reaction solvent), an acid catalyst, and a polymerization inhibitor, while removing the water formed during the reaction;
iii) neutralizing said mixture with an aqueous base;
iv) separating organic phase from said mixture;
v) removing said reaction solvent from said organic phase by evaporation, thereby obtaining a crude oily product;
vi) chromatographying said crude product, employing a second organic solvent (chromatography solvent); and
vii) removing said second solvent; thereby obtaining said monomers, having a purity of at least 95%.

2. A process according to claim 1, wherein said BBMP derivatives are selected from the group consisting of
3-bromo-2,2-bis(bromomethyl)propane-1-ol;
2,2-bis(bromomethyl)propane-1,3-diol; and
3,3′-oxybis(2,2-bis(bromomethyl)propan-1-ol).

3. A process according to claim 1, wherein said acrylic acid derivative is selected from acrylic acid, methacrylic acid, acryloyl halides.

4. A process according to claim 1, wherein said ester monomer is an acrylate monoester.

5. A process according to claim 1, wherein said ester monomer is an acrylate diester.

6. A process according to claim 1, wherein said ester monomers are selected from the group consisting of
3-bromo-2,2-bis(bromomethyl)propyl acrylate;
3-bromo-2,2-bis(bromomethyl)propyl methacrylate;
2,2-bis(bromomethyl)propane-1,3-diyl diacrylate;
2,2-bis(bromomethyl)propane-1,3-diyl bis(2-methylacrylate);
3,3′-oxybis(2,2-bis(bromomethyl)propane-1-yl) diacrylate; and
3,3′-oxybis(2,2-bis(bromomethyl)propane-1-yl) bis(2-methylacrylate).

7. A process according to claim 1, wherein removing water in said step (ii) comprises azeotropic distillation.

8. A process according to claim 1, wherein said reaction solvent is selected from the group of aliphatic and aromatic hydrocarbons.

9. A process according to claim 1, wherein said chromatography solvent is a non polar solvent which may be the same or different from reaction solvent.

10. A process according to claim 1, wherein said crude product of said step (iv) is subjected to at least one repeated flash chromatography.

11. A process according to claim 1, wherein the temperature throughout said process does not exceed 100° C.

12. A process according to claim 1, wherein said catalyst is sulfuric acid.

13. A process according to claim 1, wherein said polymerization inhibitor is selected from p-methoxyphenol, hydroquinone, and sulfur.
